(19) United States
(12) Patent Application Publication
(10) Pub. No.: US 2026/0354525 A1
(43) Pub. Date: Nov. 12, 2026

(54) ORTHOTIC STIMULATING NATURAL FOOT BIOMECHANICS

(71) Applicant: Tomer Moshe GILBOA, SHEKHANYA (IL)

(72) Inventor: Tomer Moshe GILBOA, SHEKHANYA (IL)

(21) Appl. No.: 19/298,668

(22) Filed: Aug. 13, 2025

(30) Foreign Application Priority Data
May 8, 2025 (IL) 320796

(51) Int. Cl. A43B 7/24; A61B 5/103; A61F 5/14

(52) U.S. Cl. CPC A43B 7/24; A61F 5/14; A61B 5/1038

(57) ABSTRACT

The orthotic, according to aspects of the present invention, is intended to replicate an uneven, sometimes undulating surface similar to the natural environments on which our feet have evolved to function, thereby activating the foot, knee, and pelvic joints in a more natural manner. The orthotics of the present invention may be used, among other things, for treatment, pain relief, and correction of excessive pronation and flat feet. An orthotic according to some aspects of the present invention includes a first raised area positioned against the foot heel; a second raised area positioned against the head of the first metatarsal bone; and a third raised area positioned against the head of the fifth metatarsal bone. The raised areas create a concavity at the orthotic center. This structure functions to influence the gait pattern. It encourages both pronation and the resultant supination, and helps to widen the foot's points of contact with the ground.

### CROSS-REFERENCES TO RELATED APPLICATIONS

This application claims priority of the Israeli patent application No. 320796, filed on May 8, 2025, entitled ORTHOTIC STIMULATING NATURAL FOOT BIOMECHANICS by Dr Tomer Moshe GILBOA.

### TECHNICAL FIELD

The present invention is related to foot orthopedics, more specifically, the present invention is related to orthotic.

### BACKGROUND

Pronation is a natural movement of the foot that occurs during foot landing while running or walking. It consists of three cardinal plane components with opposing motions in different parts of the foot:
Rearfoot: eversion, plantarflexion, and adduction (internal rotation).
Midfoot and forefoot: inversion, dorsiflexion, and abduction (external rotation).
Toes: eversion, plantarflexion, and abduction (external rotation).

Pronation is a normal, desirable, and necessary part of the gait cycle. It plays a crucial role in preparing the foot for push-off and absorbing impact forces. Pronation occurs during the first half of the stance phase, while supination initiates the propulsive phase as the heel begins to lift off the ground.

However, excessive pronation-when the foot rolls inward and the arches flatten more than normal-can lead to biomechanical imbalances. This condition may be influenced by genetic predisposition, weak foot muscles, improper footwear, and repetitive stress from high-impact activities. Additionally, modern lifestyles contribute to excessive pronation. Unlike our ancestors, who walked and ran barefoot on uneven natural terrain, most people today wear shoes and move primarily on hard, flat surfaces such as sidewalks and floors. This artificial environment reduces the foot's natural ability to strengthen and adapt, potentially leading to excessive pronation and related musculoskeletal issues.

Modern orthotics are primarily designed to stabilize the foot and address excessive pronation, helping to restore proper alignment and reduce strain on the musculoskeletal system.

Examples of these current techniques can be found in the following sources, accessed on Jan. 22, 2025:
What Are Orthotics and Why Should You Use Them?-Neufoot [https://www.neufoot.com/blog/what-are-orthotics-and-why-should-you-use-them]
Custom-Made Orthotics and Insoles—Ariel Comfort [https://ariel-orthopedic.co.il/custom-made-orthotics-and-custom-insoles-ariel-comfort/]
Oron Medical Orthotics (English) [https://www.oronm.co.il/english]
Five Facts About Orthotics-Avidor Healthcare [https://www.avidorhc.co.il/en/orthotics/five-facts-about-orthotics/]

Although effective in achieving stability, this method comes with drawbacks: it restricts the foot's full range of motion. By counteracting pronation, orthotics also limit supination—an essential movement for maintaining proper joint function. When pronation is restricted, joints remain closed, preventing muscle elongation. Without adequate stretching, muscles cannot contract effectively, ultimately compromising joint mobility.

These limitations extend beyond the feet. The human body operates as a cohesive kinetic chain, meaning that restricted foot motion can influence the entire system, reducing overall mobility and potentially affecting distant regions of the body.

Ordinal numbers, such as ‘first,’ ‘second,’ and ‘third,’ may alternatively be written as ‘1st,’ ‘2nd,’ and ‘3rd’ hereinafter, unless otherwise specified.

The terms ‘main body’ and ‘base’ may be used interchangeably hereinafter.

In the context of the present invention, the term ‘wedge’ shall refer to a tool, typically characterized by one or more sloping surfaces, utilized during orthopedic foot assessment. It is understood that a wedge, in its general form, presents a shape having at least one inclined plane or surface, which may converge with another surface or edge. Conversely, the term ‘post’ shall denote a structural element incorporated into the orthotic during its fabrication, which may also present one or more sloping surfaces or wedge-like features. It is to be understood that while the terms ‘wedge’ and ‘post’ are employed herein to distinguish between the assessment tool and the orthotic structural element, respectively, the ‘wedge’ may be used in the field for both the tools used in the foot assessment and the structural element. It is to be understood however that the scope of the invention is not limited to these specific shapes. Other geometric configurations that achieve substantially the same function are within the purview of the present application.

### SUMMARY OF EMBODIMENTS OF THE INVENTION

The present invention seeks to overcome the limitations of existing techniques. The orthotic, according to aspects of the present invention, is intended to replicate an uneven, sometimes undulating surface similar to the natural environments on which our feet have evolved to function, thereby activating the foot, knee, and pelvic joints in a more natural manner.

The orthotics of the present invention may be used, among other things, for treatment, pain relief, and correction of excessive pronation and flat feet.

An orthotic according to some aspects of the present invention includes a first raised area positioned against the foot heel; a second raised area positioned against the head of the first metatarsal bone; and a third raised area positioned against the head of the fifth metatarsal bone. The raised areas create a concavity at the orthotic center. This structure functions to influence the gait pattern. It promotes the natural biomechanics of the foot. It encourages both pronation and the resultant supination, and helps to widen the foot's points of contact with the ground. An orthotic embodying the invention includes a base sloped inwards and forwards from the posterolateral edge of the heel toward the anteromedial edge; and one or more posts positioned in any combination of the sagittal, frontal, and transverse planes. The orthotic may include a slope angling inwards from the metatarsal heads (ball joint area) area toward the heel area. The raised area positioned against the head of the first metatarsal bone may be sloped from the orthotic medial border toward the orthotic center. The raised area positioned against the head of the fifth metatarsal bone may be sloped from the orthotic lateral border toward the orthotic center. In some embodiments, the raised areas positioned against the head of the first metatarsal bone, the head of the fifth metatarsal bone, and the heel, may each or any combination thereof include one or more posts positioned in any combination of the sagittal, frontal, and transverse planes. To remove any doubt for the reader, the term ‘any combination of the sagittal, frontal, and transverse planes’, referenced above encompasses posts positioned within one, two, or all three planes.

Orthotic according to some aspects of the present invention is produced by the steps of fixing one or more posts to an orthotic base, positioned under one or more of the following foot anatomical parts: the heel, the head of the first metatarsal bone, the head of the fifth metatarsal bone, or any combination of these; cutting and shaping the orthotic base and the posts to conform to a predefined perimeter and profile; and applying one or more top layers and smoothing edges. In some embodiments, one or more of the positions is aligned with at least one plane of the sagittal, frontal, or transverse planes, or any combination of those planes. The posts' slopes and positions may correspond to the wedges used in an assessment procedure for which a positive result is achieved. In some examples the assessment procedure includes placing one or more of the wedges in at least one position under at least one of said anatomical parts; and recording at least one of the wedges slopes and corresponding positions resulting in a positive assessment result.

The assessment protocol is repeated both statically and dynamically, with adjustments to the depth and slopes of the wedges. In some embodiments the positive result is determined by steps of observing feet, knees, and pelvis in a resting stance; identifying foot-ground contact areas; observing foot, knees, and pelvis biomechanical function during passive and active pronation and supination; and observing relative bone movement. In some embodiments, the observation of bone movement is used to assess the biomechanical function of the foot, knee, and pelvis during passive and active pronation and supination.

### BRIEF DESCRIPTION OF THE DRAWINGS

A complete understanding of the present embodiments and the advantages and features thereof will be more readily understood by reference to the following detailed description when considered in conjunction with the accompanying drawings. Embodiments of the present disclosure are illustrated as examples and are not limited by the figures of the accompanying drawings, in which like references may indicate similar elements and in which:

FIG. 1 schematically illustrates optional placements of posts along different planes in an orthotic, according to some embodiments of the present invention.

FIG. 2A schematically illustrates the placement of the wedges along the sagittal plane during the assessment, according to some embodiments of the present invention.

FIG. 2B schematically illustrates the placements of the wedges along the frontal plane during the assessment, according to some embodiments of the present invention.

FIG. 2C schematically illustrates the placements of the wedges along the transverse plane during the assessment, according to some embodiments of the present invention.

FIG. 3 schematically illustrates an optional placement of posts along different planes in an orthotic, according to some embodiments of the present invention.

### DETAILED DESCRIPTION OF EMBODIMENTS OF THE INVENTION

Orthotics today are primarily designed to stabilize the foot and prevent pronation, the natural lowering of the three foot arches. To create these devices, a patient's foot is either scanned or cast, and an orthotic is custom-made to provide a supportive surface that evenly distributes pressure across the plantar region.

While this approach achieves its goal of stabilization, it comes at a cost: the full spectrum of natural foot movement is restricted. By preventing pronation, orthotics also inhibit supination, a movement essential for healthy joint mechanics. Without pronation, joints cannot open, which means muscles cannot stretch. The lack of muscle stretch impedes contraction, ultimately limiting joint movement.

This chain reaction doesn't stop at the feet. The body functions as an interconnected kinetic chain, and restricted foot mobility can cascade upwards, reducing overall movement capacity and affecting other parts of the body.

In contrast to existing techniques, the present invention is intended to change the gait pattern. It promotes the natural biomechanics of the foot, even when the patient is wearing shoes and walking on flat surfaces. It encourages both pronation and the resultant supination, and helps to widen the foot's points of contact with the ground.

The specific details of the single embodiment or variety of embodiments described herein are to the described system and methods of use. Any specific details of the embodiments are used for demonstration purposes only, and no unnecessary limitations or inferences are to be understood thereon. Before describing in detail exemplary embodiments, it is noted that the embodiments reside primarily in combinations of components and procedures related to the system. Accordingly, components have been represented, where appropriate in the drawings, showing only those details that are pertinent to understanding the embodiments of the present disclosure so as not to obscure the disclosure with details that will be readily apparent to those of ordinary skill in the art having the benefit of the description herein.

As an adaptation to walking on uneven natural surfaces, the foot bones evolved into a wide tripod structure formed by the heel and the heads of the 1st and 5th metatarsal bones. This anatomical configuration underpins the biomechanics of the foot in its natural function.

The orthotic, according to aspects of the present invention, is intended to restore the biomechanics associated with the normal tripod foot structure.

An orthotic, according to some aspects of the invention, includes a recess in its center. As used herein, the ‘orthotic center’ refers to the point where the three raised sections of the orthotic would intersect if connected by lines extending toward the center. It may also refer to the midpoint of the orthotic or the area positioned beneath the highest part of the foot.

In contrast to existing techniques, an orthotic according to some embodiments of the present invention incorporates raised areas beneath the heel and the heads of the 1st and 5th metatarsal bones, creating a concavity at the center of the orthotic. This allows the joints on the plantar surface of the foot—and consequently the foot's three arches—to open and spread. Such movement stimulates the proprioceptors in the soft tissues and activates the foot to function as it naturally would with a proper tripod structure. This has a positive effect on the feet, encouraging pronation and thereby triggering a supinatory response.

The orthotic according to aspects of the invention is intended to stimulate the foot's natural biomechanics. Below, we provide a more detailed explanation of how the orthotic works.

An orthotic according to some embodiments of the invention allows contact with the heads of the 1st and 5th metatarsal bones, thereby promoting pronation (lowering of the arch). Specifically, when the patient bears weight on the orthotic, the joints on the underside of the foot open more than they typically would without the orthotic. This encourages the expansion of the lateral, medial, and transverse arches of the foot, which, in turn, stretches the plantar tissue spanning these joints. The stretching induces a natural eccentric contraction of the tissue, followed by a concentric contraction, which then closes the joints. This sequential process activates the foot's natural biomechanical mechanism of supination (raising of the arch). Additionally, the raised areas under the heads of the 1st and 5th metatarsal bones create a wider base of support for weight-bearing. This engages more bones, joints, and muscles during stance and gait. As a result, the orthotic provides a more stable foundation for the entire body, reducing reliance on negative biomechanical compensatory patterns.

The orthotic may incorporate varying densities to generate elastic recoil, mimicking the natural stretch and contractile properties of muscles. In some examples, these varying densities are achieved by printing TPU with different densities. This multi-density structure also enhances shock-absorbing properties. Hence, the orthotic may support the overall functioning of the body as a biomechanically interconnected system.

In some embodiments of the invention, the orthotic consists of a main body (base) and multiple layers on top. The main body is made of TPU (thermoplastic polyurethane), which can be 3D-printed. The density of the TPU can vary depending on the patient's weight, activity type, activity frequency, and personal preferences. The main body features a slope angled inwards from the posterolateral edge of the heel toward the anteromedial edge. It incorporates three posts positioned in the sagittal, frontal, and transverse planes. Additionally, there is a slope angling inwards from the metatarsal heads (ball joint area) toward the heel. Under the head of the first metatarsal bone (big toe ball joint), there is a raised area that slopes from the medial border toward the center of the orthotic. Similarly, under the head of the fifth metatarsal bone (small toe ball joint), there is a raised area sloping from the lateral border toward the center. Both raised areas include three integrated posts positioned in the sagittal, frontal, and transverse planes. The raised areas beneath the heel and the bases of the heads of the first and fifth metatarsal bones create a concavity in the center of the orthotic. The precise positioning and slopes of the posts are determined based on the patient's foot measurements and the therapist's assessment. According to some embodiments, the slope is relative to the orthotic base; alternatively, the slope is relative to the floor.

The main orthotic body can be covered with various layers for enhanced functionality. For example, Poron may be used for shock absorption, and EVA (ethylene-vinyl acetate) may be added on top for durability, protecting the more fragile Poron layer. These layers are adhered using glue, and a vacuum pump ensures optimal adhesion.

The materials, number of layers, and the thickness of each layer can be customized according to the patient's weight, activity type, activity frequency, and personal preferences. Additionally, the orthotic can be manufactured in any color.

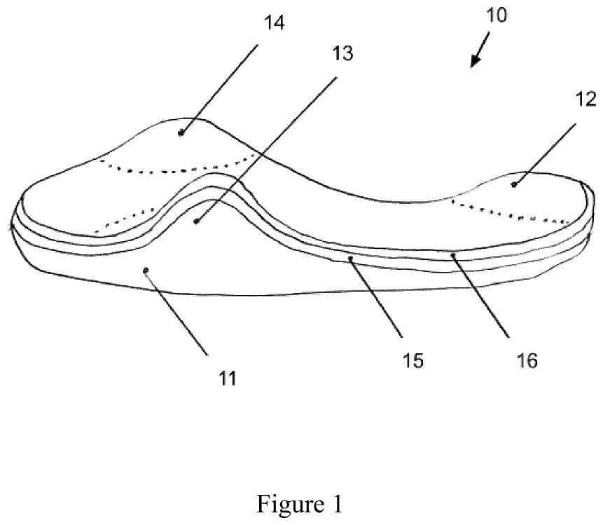

Orthotic 10, embodying the invention, is depicted in FIG. 1. Flat base 11 may have a thickness of 1 mm. Base 11 is intended to be flexible, allowing for the natural movement of the foot bones and ensuring compatibility with the foot's biomechanics. Three raised areas are shown: one under the heel 12 and two under the heads of the heads of the 1st 13 and 5th 14 metatarsal bones. These raised areas create a concavity in the center of the orthotic. The top layer of the orthotic may consist of shock absorption layer 15 and friction-resistant layer 16.

### Example of Orthotic Preparation Embodying the Invention

### Assessment Protocol

The biomechanical functional capacity of the patient's feet is assessed using the following protocol:
The therapist observes and notes the appearance of both feet, knees, and pelvis in a resting stance.
Palpation is performed to identify which parts of the feet make contact with the ground.
The therapist observes and records the biomechanical function during both passive (static) and active (dynamic) pronation (arch lowering) and supination (arch rising) for the feet, knees, and pelvis, with emphasis on foot-to-ground contact.
Palpation, using fingers to provide direct feedback, is used to gauge the relative movement of bones during pronation and supination. This includes manual assessment of: (1) whether a bone is moving at all, (2) the movement of a bone relative to another, and (3) the movement of neighboring bones away or towards each other, indicating joint opening or closing. These assessments are conducted in three dimensions across the foot, knee, and pelvis, with emphasis on foot-to-ground contact.
Wedges, typically made of EVA or other suitable materials, are strategically positioned under specific locations, such as the posterolateral edge of the heel and the heads of the 1st and 5th metatarsal bones. Angled inward toward the foot's center, these help influence biomechanical movements by allowing areas of the foot that are not in contact with the ground to bear weight.

The assessment protocol is repeated both statically and dynamically, with adjustments to the depth and slopes of the wedges.

A positive result is considered when there is an improvement toward proper foot and knee pronation biomechanics in weight-bearing—meaning alignment and motion within a healthy, functional range—as well as in static posture and in the dynamic biomechanics of pronation and supination of the foot, knee, and pelvis. In some embodiments, the assessment is conducted in the three cardinal planes: sagittal, frontal, and transverse.

The placements and slopes of the wedges when a positive result is achieved are recorded and used for the fabrication of the orthotic. Specifically, for the placements and slopes of the posts. In some embodiments, the wedge slope is relative to the floor. Post slopes are relative to the orthotic base. By way of non-limiting example, the post slope angle varies according to patient needs, with average angles ranging from approximately 4 degrees to 30 degrees. Similarly, while dependent on patient foot size, the wedges' and posts' typical widths may range from approximately 4 cm to 15 cm, with an average of approximately 6 cm.

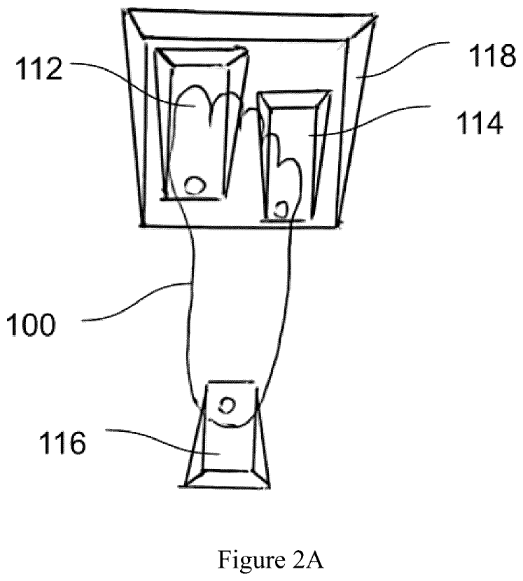

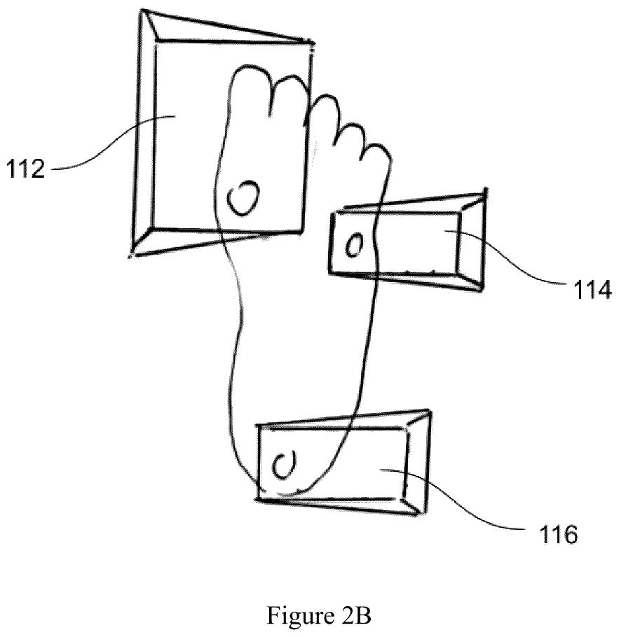

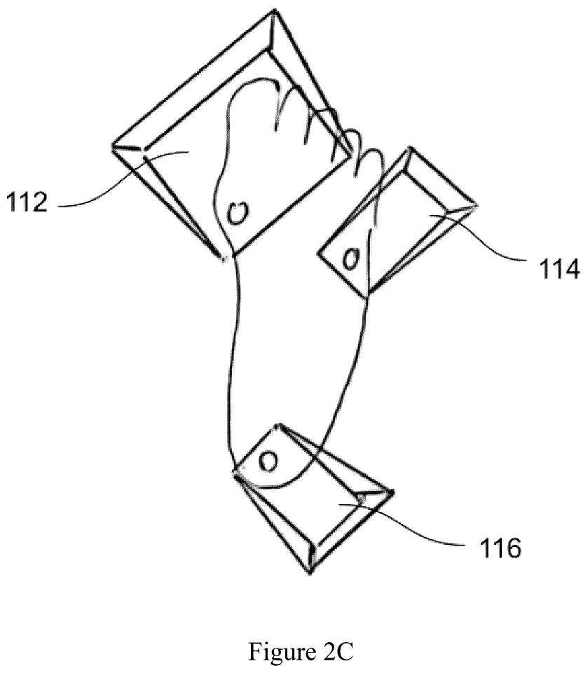

The placements of the wedges along the sagittal, frontal, and transverse planes during the assessment of right foot 100, according to some embodiments of the present invention, are schematically depicted in FIGS. 2A, 2B, and 2C, respectively. Wedge 112 is placed under the location of the head of the 1st metatarsal bone, while wedges 114 and 116 are placed under the locations of the head of the 5th metatarsal bone and the heel, respectively. The purpose of wedge 118, shown in FIG. 2A, is to increase the slopes of both wedges 112 and 114, which are placed on top of wedge 118.

### Orthotic Fabrication

The feet are scanned using a foot scanner (e.g., USOL-3D), and the software identifies the locations of the heel, and the heads of the 1st and 5th metatarsal bones.
Optionally, for human (therapist) verification, stickers can be placed on the underside of the foot at these locations. These stickers act as alignment markers, allowing the therapist to verify the software's accuracy.
The software (e.g. ISOLECAD-X) generates a flat template for the orthotic base. Sloping posts are added at positions under the heel and the heads of the 1st and 5th metatarsal bones. These posts form the elevated (raised) areas of the orthotic discussed herein. The positions, sizes, and slopes of the posts are determined based on assessment results and measurements. The posts may be positioned in any plane of motion, such as the sagittal, frontal, or transverse planes.

### 3D Printing and Post-Processing

Printing: The orthotic is printed using a 3D printer (e.g. ISOLEPRINT-X2) in densities tailored to the patient's weight, activity type, activity frequency, and personal preferences.
Smoothing: After printing, rough edges are smoothed using a grinding/milling machine such as ISOLETEK Orthotic Grinder to create a comfortable surface.
Top Layers: Layers of Poron (for shock absorption) and EVA (for durability and comfort) are glued onto the orthotic's top surface using textile glue. The number of layers and their thickness (2-3 mm) depend on the patient's specific needs.
Vacuum Adhesion: A pump vacuum may be applied to ensure seamless adhesion of the layers.
Finishing: Excess material is trimmed with scissors, and the edges are polished with a grinder to achieve a smooth, flush finish.

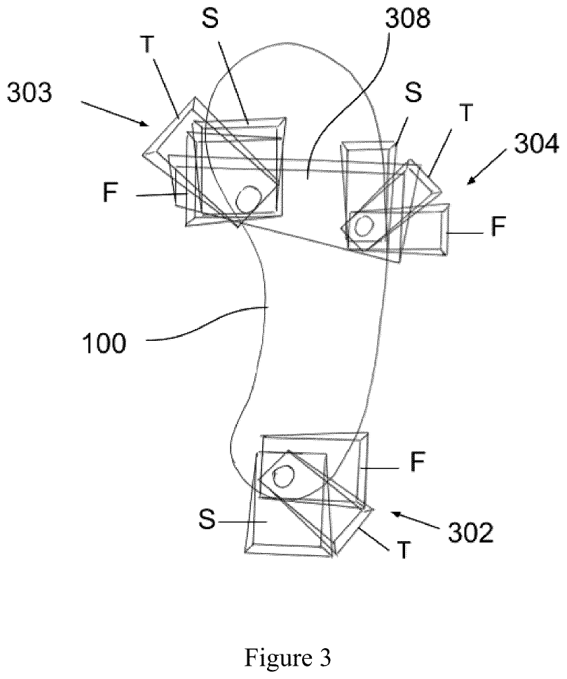

Optional positions of the posts, according to some embodiments, during the fabrication of the orthotic are depicted schematically in FIG. 3. Posts 302, 303, and 304 are positioned under the heel, and the heads of the 1st and 5th metatarsal bones of the right foot 100, respectively. Additionally, FIG. 3 shows post 308 located beneath posts 303 and 304 to further increase the rise of the heads of the 1st and 5th metatarsal bones, compared to the rise caused by posts 303 and 304 alone. The optional positions shown are along the sagittal, frontal, and transverse planes, marked ‘S’, ‘F’, and ‘T’, respectively. It should be understood that the above posts are cut to conform to the orthotic perimeter in the subsequent steps of fabrication or post-processing.

It should be understood that elements and/or features of an apparatus, or a method described herein can be combined in a variety of ways without departing from the spirit and scope of the present teachings, whether explicit or implicit herein. For example, where reference is made to a particular structure, that structure can be used in various embodiments of apparatus of the present teachings and/or in methods of the present teachings, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

The use of the singular herein, for example, “a,” “an,” and “the,” includes the plural (and vice versa) unless specifically stated otherwise.

The use of any and all examples, or exemplary language herein, for example, “such as,” “including,” or “for example,” is intended merely to better illustrate the present teachings and does not pose a limitation on the scope of the invention unless claimed. No language in the specification should be construed as indicating any non-claimed element as essential to the practice of the present teachings.

The present teachings encompass embodiments in other specific forms without departing from the spirit or essential characteristics thereof. The foregoing embodiments are therefore to be considered in all respects illustrative rather than limiting on the present teachings described herein.

1. An orthotic comprising:
a first raised area positioned against the foot heel;
a second raised area positioned against the head of the first metatarsal bone; and
a third raised area positioned against the head of the fifth metatarsal bone.

2. The orthotic of claim 1, wherein said raised areas create a concavity at said orthotic center.

3. The orthotic of claim 1 comprising:
a base sloped inwards and forwards from the posterolateral edge of the heel toward the anteromedial edge; and
at least one post positioned in planes selected from the group consisting of sagittal, frontal, and transverse and combinations thereof.

4. The orthotic of claim I comprising a slope angling inwards from the metatarsal heads (ball joint area) area toward the heel area.

5. The orthotic of claim 1 wherein said raised area positioned against the head of the first metatarsal bone is sloped from said orthotic medial border toward said orthotic center.

6. The orthotic of claim 1 wherein said raised area positioned against the head of the fifth metatarsal bone is sloped from said orthotic lateral border toward said orthotic center.

7. The orthotic of claim I wherein said raised area positioned against the head of the first metatarsal bone comprises at least one post positioned in planes selected from the group consisting of sagittal, frontal, and transverse and combinations thereof.

8. The orthotic of claim 1 wherein said raised area positioned against the head of the fifth metatarsal bone comprises at least one post positioned in planes selected from the group consisting of sagittal, frontal, and transverse and combinations thereof.

9. The orthotic of claim I wherein said raised area positioned against the heel comprises at least one post positioned in planes selected from the group consisting of sagittal, frontal, and transverse and combinations thereof.

10. An orthotic produced by the steps of:
fixing three posts to an orthotic base at positions under the foot heel, the head of the first metatarsal bone, and the head of the fifth metatarsal bone;
cutting and shaping said orthotic base and said posts to conform to a predefined perimeter and profile to form first, second, and third raised areas at said positions; and
applying a top layer and smoothing edges.

11. The orthotic of claim 10 wherein at least one of said positions is aligned with at least one plane selected from the group consisting of sagittal, frontal, transverse, and combinations thereof.

12. The orthotic of claim 10, wherein said posts slopes and positions correspond to wedges used in an assessment procedure for which a positive result is achieved, said procedure comprising the steps of:
placing at least one of said wedges in at least one position under at least one of said anatomical parts; and
recording at least one of said wedges slopes and corresponding positions resulting in a positive result.

13. The orthotic of claim 12 wherein said positive result determined by steps of:
observing feet, knees, and pelvis in a resting stance;
identifying foot-ground contact areas;
observing foot, knees, and pelvis biomechanical function during passive and active pronation and supination; and
observing relative bone movement.
